(19) United States
(12) Patent Application Publication
(10) Pub. No.: US 2022/0096686 A1
(43) Pub. Date: Mar. 31, 2022

(54) Pathogen Inactivation by Exposure to a Noble Gas

(71) Applicant: Robert H. Hodam, Elk Grove, CA (US)

(72) Inventor: Robert H. Hodam, Elk Grove, CA (US)

(21) Appl. No.: 17/039,761

(22) Filed: Sep. 30, 2020

(51) Int. Cl. A61L 2/20

(52) U.S. Cl. CPC A61L 2/20; A61L 2202/14; A61L 2202/24; A61L 2202/122

(57) ABSTRACT

A method for inactivating and/or destroying microscopic biological pathogens includes placing an object to be treated in a pressure vessel, exposing the object to a pressurized noble gas at a selected target treatment pressure for a selected treatment time duration, then decreasing the treatment chamber pressure from the target treatment pressure to external atmospheric pressure within a selected depressurization time duration. The method has been tested effective against pathogens for target treatment pressure in a range from 60 psi to 150 psi and treatment time duration in a range from 300 seconds to 600 seconds.

### FIELD OF THE INVENTION

Embodiments are related in general to methods for destroying pathogens present on inanimate objects and more specifically to methods for exposing biological pathogens to a gas pressurized above atmospheric pressure.

### BACKGROUND

Medical instruments, implants, laboratory equipment, and other materials and items to be brought into contact with a person or which have already been in contact with a person may be disinfected to prevent the transmission of diseases and infections caused by biological pathogens. Biological pathogens, for example microscopic forms such as bacteria, bacterial spores, archaea, protozoa, fungi, and viruses may be inactivated or destroyed by exposure to disinfecting agents. Disinfecting agents such as chemical compounds, electromagnetic radiation, heat, and high-energy particles such as electrons, protons, and alpha particles may be used individually or in combination with one another to inactivate, destroy, and/or remove pathogens.

Each of the previous examples of disinfecting agents has disadvantages. For example, a chemical compound effective against one type of pathogen may not be effective against other types of pathogens. Chemical compounds used as disinfecting agents may participate in chemical reactions that weaken, erode, dissolve, discolor, or affect other material properties of an object being disinfected. Biological pathogens may develop resistance to a chemical compound used as a disinfecting agent. Chemical reaction byproducts or residual amounts of a chemical disinfecting agent may render a treated object unfit for use or may be sufficiently toxic to present a safety hazard to people performing disinfecting procedures or using disinfected objects. Occupational diseases are associated with some chemical disinfecting agents, for example formaldehyde, chlorine, and others. Some chemical disinfecting agents, for example hydrogen peroxide, may cause skin and eye irritation and may break down violently if stored improperly. Some chemical disinfecting agents, for example gas mixtures including ethylene oxide, may be used for disinfecting objects which are preferably not to be exposed to high temperatures or moisture. However, ethylene oxide presents a combustion hazard and/or an explosion hazard. Exposure to ethylene oxide has also been reported by the United States Department of Labor to be associated with respiratory irritation, lung injury, neurotoxicity, cancer, and other medical problems.

Electromagnetic radiation such as blue light, ultraviolet light, x-rays, and microwaves have been used to destroy microorganisms. The wavelength and intensity of electromagnetic radiation used for disinfection are selected according to the type of pathogen to be destroyed and the chemical, mechanical, and optical properties of an object to be disinfected. The particular frequency, intensity, and/or duration of electromagnetic radiation directed at the object may fail to destroy pathogens. High-energy particles may have similar limitations. Shadowed areas and other parts of an object not exposed to sufficient electromagnetic radiation or high-energy particles can be reservoirs for biological pathogens. Electromagnetic radiation and/or high-energy particles may alter the material properties of an object, reduce the utility or service lifetime of the object, or cause burning, melting, or other forms of structural damage to the object.

Exposing objects to be disinfected to high temperatures has long been known to be effective for destroying biological pathogens. However, temperatures sufficiently high to destroy pathogens may melt or deform some materials or cause materials such as paper or plastic to combust. High temperatures may damage or weaken glassware and other materials through the effects of thermal expansion, cause closed containers or materials with closed cavities to rupture or explode, or promote chemical breakdown, for example by damaging polymer materials used in liquid or gas seals. Autoclaves and other equipment used for exposing items to high temperatures have exploded from internal overpressure, caused fires from attempts to disinfect flammable and/or volatile materials, and caused thermal burns to persons contacting the equipment or treated objects. Steam injected into a chamber holding objects to be disinfected may damage the objects by water absorption or chemical attack and presents a significant risk of severe thermal burns to nearby persons.

Substantial research has been performed to evaluate inactivation of pathogens by exposure to a pressurized gas such as carbon dioxide, nitrogen, oxygen, and/or argon. Pathogens have been exposed to one or more of these gases at pressures substantially higher than atmospheric pressure for durations of tens of minutes to many hours, followed by depressurization and a return to normal atmospheric pressure. Rupturing of cell walls or virus envelopes has been reported as a result of rapid depressurization. Slower depressurization has been reported to interfere with molecular structures and/or biochemical reactions in some pathogens. A rate of diffusion of the pressurized gas through the cell walls or virus envelopes has been proposed as the rate limiting step, with longer exposure times to pressurized gas correlating to greater inactivation or destruction of pathogens. Inactivation or destruction of pathogens has been reported to be improved by subjecting the pathogens to multiple pressurization/depressurization cycles of the tested gases. Some results have indicated that for a test group consisting of carbon dioxide, nitrogen, oxygen, and argon, carbon dioxide was the most effective for inactivation or destruction of pathogens under the conditions of the tests. However, some studies have shown that carbon dioxide is sometimes ineffective for inactivating or destroying bacterial spores and some other biological pathogens.

### BRIEF DESCRIPTION OF THE DRAWINGS

FIG. 1 shows a schematic diagram of an example apparatus configured for use with the disclosed method embodiments.

FIG. 2 shows an example of one complete pressure treatment cycle in accord with the disclosed method embodiments, presented as a graph of changes in noble gas pressure in a treatment chamber as a function of time.

### SUMMARY

An example embodiment of a method for inactivating biological pathogens includes selecting a treatment time duration, a target treatment pressure, and a depressurization time duration. The example method further includes placing an object in a treatment chamber of a pressure vessel; and closing the treatment chamber, an exhaust valve, and a depressurization valve. After closing the treatment chamber, the example method further includes opening an inlet valve and introducing a noble gas into the treatment chamber; increasing a treatment chamber pressure to the target treatment pressure; and holding the treatment chamber pressure constant at the target treatment pressure for the treatment time duration. After the treatment time duration, the example method further includes opening the depressurization valve sufficiently to decrease the treatment chamber pressure to the external atmospheric pressure.

The example method further includes any one or more of the following operations, individually or in any combination or subcombination: selecting helium as the noble gas; selecting neon as the noble gas; selecting argon as the noble gas; selecting the value of the treatment time duration in a range from 300 seconds to 600 seconds; selecting the value of target treatment pressure in a range from 60 pounds-force per square inch (psi) to 150 psi; and selecting the value of depressurization time duration in a range of 0.5 second to 3.0 seconds.

For the disclosed example method embodiments, the treatment chamber pressure is less than the target treatment pressure and greater than the external atmospheric pressure during the depressurization time duration and approximately equal to the external atmospheric pressure after the depressurization time duration, and the noble gas is selected to have a molecular weight less than or equal to a molecular weight of argon.

### DESCRIPTION

Example embodiments of a method for inactivating biological pathogens include placing one or more inanimate objects to be treated in a treatment chamber of a pressure vessel, sealing the vessel to prevent the escape of gas from the treatment chamber, and exposing the objects in the treatment chamber to a noble gas having a selected target treatment pressure substantially higher than atmospheric pressure external to the pressure vessel. Exposure at the target treatment pressure preferably continues for a treatment time duration sufficient for the noble gas to diffuse through the cell walls or outer envelopes of biological pathogens on or in the objects. After exposing the objects to the pressurized noble gas for the selected treatment time, the pressure of the noble gas in the treatment chamber is rapidly reduced to external atmospheric pressure within a selected depressurization time duration that is substantially shorter than the treatment time duration. The noble gas that diffuses into the pathogens inactivates or destroys the pathogens when the treatment chamber is rapidly depressurized.

The disclosed method embodiments are effective for inactivating and/or destroying biological pathogens including, but not limited to, amoeba, paramecia, bacteria, archaea, protozoa, fungi, viruses, and bacterial spores. The disclosed method embodiments disinfect objects more quickly, with less damage to the objects being treated, and with fewer safety hazards than previously known methods using carbon monoxide, carbon dioxide, chlorine, hydrogen peroxide, ethylene oxide, high temperatures, steam, and/or radiation. For example, unlike chemically inert noble gases, carbon monoxide may interact chemically with pathogens, thereby decreasing an amount of the gas in a pressure vessel effective for inactivating pathogens by rapid depressurization. Further unlike noble gases, flammability and toxicity hazards are associated with carbon monoxide and other gases previously used to destroy pathogens.

The disclosed method embodiments are effective for inactivating and/or destroying microscopic biological pathogens present on or in objects such as, but not limited to, surgical instruments, bandages, surgical dressings, suture materials, respirators, face masks, catheters, medical implants, gloves, gowns, trays, pans, specimen containers, optically transparent eye and/or face shields, fiber optic instruments, optical components with materials susceptible to damage by common disinfecting agents and/or water, instruments with electronic components, food items intended for human consumption, and other items which are preferably not to be exposed to high temperatures, liquid water, solvents, steam, oxidizers, strong acids (low pH), strong bases (high pH), explosive and/or flammable chemical compounds, chemicals toxic to humans, chemicals with an associated risk of causing cancer, and so on. Objects made from paper, polymer materials which may soften or melt when exposed to boiling water or steam, composite materials, wood, glass, ceramic, stainless steel alloys, metal alloys which may corrode or rust when exposed to water, open-cell foam materials, closed-cell foam materials, natural fibers such as cotton, flax, and hemp, synthetic fibers such as nylon and polyester, and many other materials may safely be treated by the disclosed method embodiments to inactivate and/or destroy pathogens.

For descriptive purposes herein, a biological pathogen is considered to be inactivated when the pathogen is rendered incapable of transmitting disease and/or incapable of reproducing, whether or not when the pathogen's cell wall, virus envelope, or other outer layer appears intact after treatment is complete. A biological pathogen is considered to be destroyed when the pathogen's outer layer is ruptured or otherwise structurally damaged sufficiently to prevent the pathogen from transmitting disease and/or reproducing.

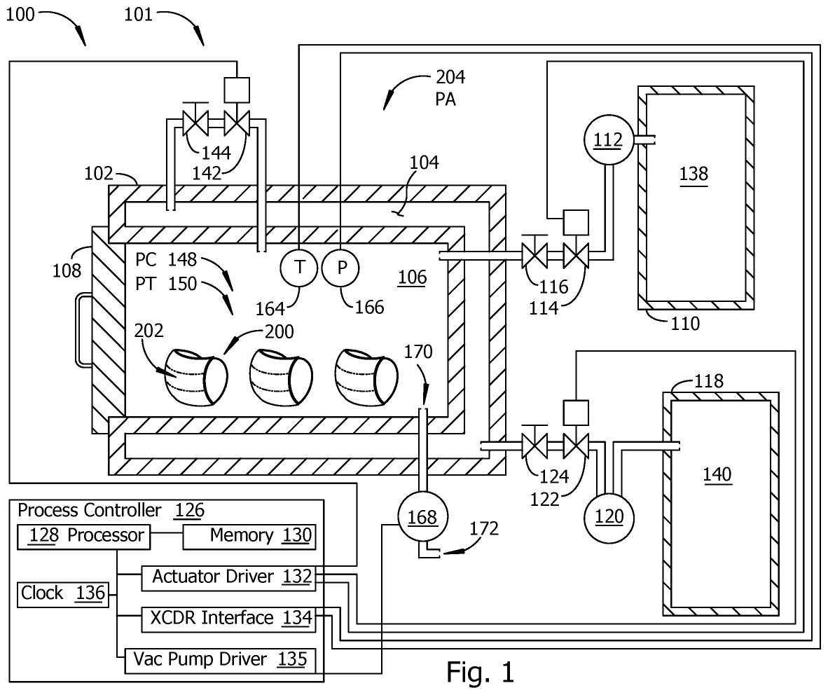

FIG. 1 illustrates a schematic of an example treatment apparatus 101 suitable for use with the disclosed method embodiments 100. Objects to be disinfected 200 which may have been contaminated with a biological pathogen 202 are placed in the treatment chamber 106 of a dual-chamber pressure vessel 102. In the example of FIG. 1, paper face masks represent examples of objects 200 to be treated for inactivation and/or destruction of biological pathogens 202 present on or in the objects. A noble gas 138 stored under pressure in a noble gas supply vessel 110 is admitted into the treatment chamber 106 after passing through one or more inlet valves (114, 116) and an optional treatment chamber pressurization pump 112. When closed by a chamber door 108, the treatment chamber is capable of being pressurized to a value PC 148 substantially above atmospheric pressure PA 204 external to the treatment chamber by the pressure of the noble gas 138 in the supply vessel 110 and/or by operation of the treatment chamber pressurization pump 112. When the noble gas in the treatment chamber 106 reaches a preferred pressure value PT 150, gas flow between the noble gas supply vessel 110 and the treatment chamber 106 may be interrupted by operation of a manually-actuated treatment chamber inlet valve 116 and/or an electrically-actuated treatment chamber inlet valve 114, either one or alternately both inlet valves optionally present in the apparatus. The noble gas supply vessel 110 may be fitted with a separate shut-off valve (not illustrated).

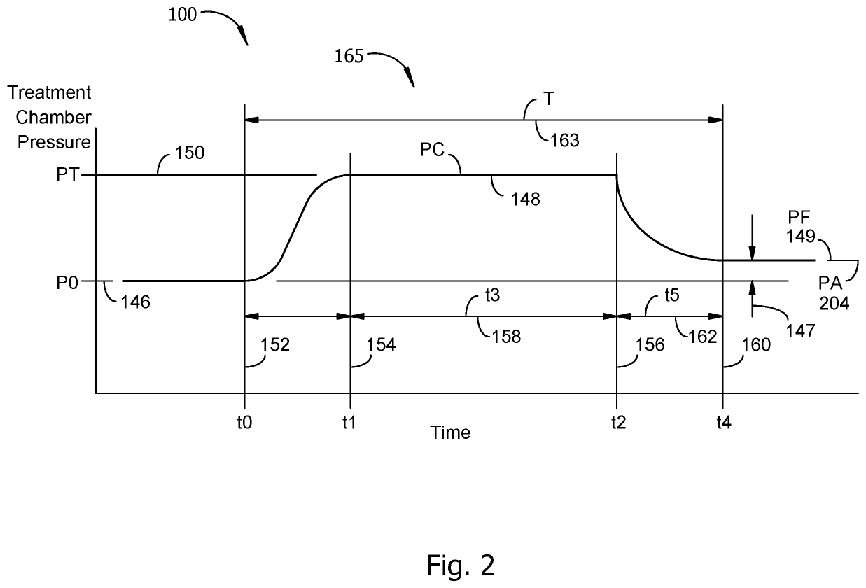

The treatment chamber 106 of the dual-chamber pressure vessel 102 is configured to be in fluid communication with an exhaust gas chamber 104 through one or more intervening depressurization valves (142, 144). In the example of FIG. 1, the exhaust gas chamber 104 surrounds the treatment chamber 106. The exhaust gas chamber 104 may alternatively be provided as a separate container not integrally formed with the treatment chamber 106. After the objects 200 in the treatment chamber 106 have been exposed to the selected target treatment pressure PT 150 of noble gas for a preferred treatment time duration (e.g. t3 158 FIG. 2), the treatment chamber may be depressurized by operation of a manually-actuated depressurization value 144 and/or an electrically-actuated depressurization valve 142, either one or alternately both depressurization valves (142, 144) optionally present in the apparatus.

When the depressurization valve(s) are opened, noble gas at high pressure flows from the treatment chamber 106 into the exhaust gas chamber 104, rapidly decreasing the pressure in the treatment chamber 106. The contents of the exhaust gas chamber 104 may be transferred into an exhaust gas recovery vessel 118 through one or more exhaust valves (122, 124) and an optional exhaust gas pump 120 in fluid communication with the exhaust gas chamber and recovery vessel. Exhaust gas may be withdrawn from the exhaust gas chamber 104 through a manually-actuated exhaust valve 124 and/or an electrically-actuated exhaust valve 122, either one or alternately both exhaust valves optionally present in the apparatus. Exhaust gas 140 in the exhaust gas recovery vessel 118 includes the noble gas 138 used to pressurize the treatment chamber 106, residual air from the treatment chamber 106, and any entrained emissions from the objects 200 being treated. Capturing the exhaust gas 140 in the recovery vessel 118 is preferable to venting the gas into the atmosphere.

The example treatment apparatus 101 may be controlled by a person operating the manually-actuated inlet valve 116, the manually-actuated depressurization valve 144, and the manually-actuated exhaust valve 124 in accord with the disclosed method embodiment 100. The treatment apparatus 101 may optionally include a process controller 126 configured to operate the pumps and valves needed to expose the objects 200 to the preferred target treatment pressure PT 150 of noble gas for the preferred treatment time duration t3 158. The example process controller 126 of FIG. 1 includes a processor 128 in data communication with a memory 130, an actuator driver 132, a transducer (XDCR) interface 134, an optional vacuum pump driver 135, and a clock circuit 136. The memory 130 stores operating instructions and data in a form accessible to the processor 128. The actuator driver 132 is configured to send electrical signals to the electrically-actuated treatment chamber inlet valve 116, the electrically-actuated exhaust valve 122, and the electrically-actuated depressurization valve 142. The transducer interface 134 is configured to convert electrical signals from a temperature transducer 164 and a pressure transducer 166 positioned to measure conditions in the treatment chamber 106 into data accessible to the processor 128. The clock circuit 136 may be accessed by the processor 128 to establish a start time, a time duration, and/or a stop time for exposing pathogens to a noble gas for the treatment time duration 158.

The example treatment apparatus 101 optionally includes a vacuum pump 168 having a pump inlet 170 in fluid communication with the treatment chamber 106 and a pump outlet 172 in fluid communication with the external atmosphere. The vacuum pump 168 may be electrically connected to the vacuum pump driver 135 in data communication with the processor 128 or may alternatively be configured for manual activation. The process controller 126 may optionally be configured to operate the vacuum pump 168 to reduce pressure PC 148 in the treatment chamber 106 after the chamber door 108 has been closed, the depressurization valve(s) (142, 144) closed, the exhaust valve(s) (122, 124) closed, and before noble gas 138 is admitted into the treatment chamber. Treatment chamber pressure PC 148 may be determined by the processor 128 from signals from the pressure transducer 166. The process controller 126 is preferably configured to maintain treatment chamber pressure PC 148 within a range selected to avoid structural damage to the pressure vessels, piping, valves, and other parts of the apparatus 101.

Opening the chamber door 108 allows air at external atmospheric pressure PA 204 to enter the treatment chamber 106. After closing and sealing the chamber door, reducing air pressure in the treatment chamber before introducing noble gas decreases the amounts of oxygen, nitrogen, water vapor, carbon dioxide, and other components of air present in the treatment chamber 106, enabling an increase in the fraction of noble gas and a decrease in the fraction of other gases when the treatment chamber pressure PC 148 reaches the target treatment pressure PT 150. Increasing the fraction of noble gas present in the treatment chamber is advantageous for reducing a time duration T 163 of one complete pressure treatment cycle. Reducing a fraction of air in the mixture of gases captured in the exhaust gas recovery vessel 118 enables more efficient re-use and/or recycling of noble gas 138.

The example treatment apparatus 101 of FIG. 1 may be operated in accord with a method embodiment 100 to generate a pressure treatment cycle as shown in the example of FIG. 2. The graph in the example of FIG. 2 is a plot of treatment chamber pressure PC 148 in the treatment chamber 106 as a function of time for one complete pressure treatment cycle 165 of time duration T 163 for inactivating and/or destroying pathogens 202 in the treatment chamber. A complete pressure treatment cycle 165 refers to a preferred sequence of changes in the treatment chamber pressure PC 148 over a time duration T 163 from t0 152 to t4 160, as suggested in the example of FIG. 2. After closing the chamber door 108, and after fully closing the treatment chamber inlet valve(s) (114, 116), the exhaust valve(s) (122, 124), and the depressurization valve(s) (142, 144), the initial pressure in the treatment chamber PC at the start t0 of a pressure cycle is PO 146. In some method embodiments 100, the initial pressure PO 146 is equal to external atmospheric pressure PA 204 at about 14.7 pounds-force per square inch (psi) or 101 kilopascals (kPa). Alternatively, the vacuum pump 168 may be operated to reduce pressure PC 148 in the treatment chamber 106 below external atmospheric pressure before admitting noble gas 138 into the treatment chamber.

At the selected start time t0 152, the inlet valve(s) (114, 116) are opened, allowing noble gas 138 to flow from the supply vessel 110 into the treatment chamber 106. The treatment chamber pressurization pump 112 may be operated to increase the treatment chamber pressure PC 148 until the treatment chamber pressure attains the target treatment pressure PT 150. At time t1 154, the time at which PC 148 is equal to PT 150, the inlet valve(s) (114, 116) may be closed. Alternatively, the treatment chamber pressurization pump 112 may be operated to hold treatment chamber pressure PC constant. The treatment chamber pressure PC 148 is preferably maintained with PC greater than or equal to PT for the treatment time duration t3 158. The treatment time duration t3 158 is preferably chosen such that no more than one complete pressure treatment cycle 165 is needed to achieve a satisfactory result for inactivation and/or destruction of pathogens 202 on objects 200 in the treatment chamber 106. A satisfactory result may correspond to a threshold number of observed viable pathogens or a preferred fractional reduction in viable pathogens, with the fractional reduction in a range from 0.5 to 1, 1 representing total elimination of all viable pathogens.

At a time t2 156 separated from t1 154 by the preferred treatment time duration t3 158, the treatment chamber pressure PC 148 is reduced by opening the depressurization valve(s) (142, 144), allowing noble gas at pressure PT 150 to flow from the treatment chamber 106 into the exhaust gas chamber 104. Prior to opening the depressurization valve(s), the exhaust gas chamber 104 will preferably be at substantially lower pressure than the treatment chamber 106. For example, prior to opening the depressurization valves, the pressure in the exhaust gas chamber 104 may be approximately equal to external atmospheric pressure PA 204, about 14.7 psi. After opening the depressurization valve(s), the treatment chamber pressure PC 148 preferably decreases from PT 150 at time t2 156 to a pressure PF 149 at the end of the depressurization time duration at t4 160. PF 149 may be greater than PO 146 as suggested by an example pressure difference 147 in FIG. 2. PF 149 may optionally be approximately equal to external atmospheric pressure PA 204. The pressure difference 147 may alternatively have a positive value, a value of zero, or a negative value according to selected values of PO and PF.

Depending on the relative volumes of the exhaust gas chamber 104 and treatment chamber 106 and the value of PT 150, it may be necessary to open the exhaust valve(s) (122, 124) and activate the exhaust gas pump 120 to enable the treatment chamber pressure PC 148 to decrease to a preferred value of treatment chamber pressure PF 149 at the end of a pressure treatment cycle at time t4. After the time duration T 163 corresponding to one complete pressure treatment cycle 165 has elapsed, PF 149 will preferably be allowed to equilibrate with external atmospheric pressure to enable the chamber door 108 to be opened safely.

The time interval t5 162, where t5=t4−t2, corresponds to the length of time over which depressurization occurs and treatment chamber pressure falls to its preferred value PF 149 after having been held at PT 150 for the treatment time duration t3 158. For the disclosed method embodiments 100, depressurization time duration t5 162 is preferably in a range from 0.5 second (s) to 3.0 s. The target treatment pressure PT 150 is preferably in a range from 60 pounds-force per square inch (psi) to 150 psi, corresponding to 414 kPa to 1.0 MPa. Treatment time duration t3 158 is preferably in a range from 300 s to 600 s. At a target treatment pressure PT 150 of 60 psi, petri dishes containing Escherichia coli (E. coli) bacteria as a test pathogen were sterilized after exposure to helium (He) for t3=300 s, where sterilization refers to destruction of essentially all microorganisms with no detected viable microorganisms remaining. No additional benefit for the disclosed method embodiments has been found for t3>600 s, the treatment time duration at which complete sterilization was achieved for the test pathogen.

When starting from a closed treatment chamber 106 containing air at about 14.7 psi, introducing a gas at 60 psi that is at least 99% xenon by mole fraction into the treatment chamber until the chamber stabilizes at a 60 psi leads to a gas composition in the closed chamber corresponding to approximately 81% xenon and 19% air by mole fraction. The range of values for depressurization time duration t5 162 described in the previous paragraph apply to a treatment chamber composition of at least 81% noble gas and no more than 19% air (mole fraction). Increasing the fraction of noble gas in the treatment chamber reduces the treatment time to the lower end of the stated range for treatment time. The fraction of noble gas in the treatment chamber may be increased at PC 148=PT 150 by partially evacuating air from the treatment chamber before introducing the noble gas. Reducing air pressure in the treatment chamber to about 50% of external atmospheric pressure, i.e. reducing treatment chamber pressure PC 148 from about 14.7 psi to about 7 psi by operation of the vacuum pump 168, then introducing xenon at 60 psi and waiting for treatment chamber pressure to stabilize results in a gas composition in the closed chamber of about 89% xenon and 11% air by mole fraction.

Alternatively, the gas composition in the treatment chamber may be made approximately equal to the purity of the inlet noble gas by flowing noble gas into the chamber and allowing a mix of noble gas and air to flow out. The outflow may be sustained for an amount of time determined by the flow rate, pressure, and purity of the inlet noble gas, the target values for pressure and mole fraction of noble gas in the chamber, the volume of the treatment chamber, and the outlet flow rate of the mixture of noble gas and air.

Tests for the noble gases helium (He) and argon (Ar) produce similar results, i.e. sterilization of E. coli samples for 60 psi<=PT<150 psi and 300 s<=t3<=600 s. Neon, a noble gas having a molecular weight between that of He and Ar, is predicted to be as effective as He and Ar. Noble gases with molecular weights above that of Ar may require longer treatment time durations t3 and/or higher target treatment pressures PT, possibly because of expected slower diffusion of heavier noble gas atoms through cell walls or virus envelopes, compared to lighter noble gas atoms. For the disclosed method embodiments, the preferred group of noble gases therefore consists of He, Ne, and Ar.

Unless expressly stated otherwise herein, ordinary terms have their corresponding ordinary meanings within the respective contexts of their presentations, and ordinary terms of art have their corresponding regular meanings.

1. A method, comprising:
selecting a treatment time duration of 300 seconds, a target treatment pressure in a range from 60 pounds-force per square inch (psi) to 150 psi, a noble gas, and a depressurization time duration;
placing an object in a treatment chamber of a pressure vessel;
closing the pressure vessel, an exhaust valve, and a depressurization valve;
opening an inlet valve and introducing the noble gas into the treatment chamber;
increasing a treatment chamber pressure to the target treatment pressure;
holding the treatment chamber pressure constant at the target treatment pressure for the treatment time duration; and
after the treatment time duration, opening the depressurization valve sufficiently to decrease the treatment chamber pressure to an external atmospheric pressure within the selected depressurization time duration.

2. The method of claim 1, further comprising selecting the noble gas with a molecular weight less than or equal to a molecular weight of argon.

3. The method of claim 1, further comprising selecting helium as the noble gas.

4. The method of claim 1, further comprising selecting neon as the noble gas.

5. The method of claim 1, further comprising selecting argon as the noble gas.

6. (canceled)

7. (canceled)

8. The method of claim 1, further comprising selecting the depressurization time duration in a range of 0.5 second to 3.0 seconds.

9. The method of claim 1, wherein the treatment chamber pressure is less than the target treatment pressure and greater than the external atmospheric pressure during the depressurization time duration and approximately equal to the external atmospheric pressure after the depressurization time duration.

10. The method of claim 1, further comprising reducing the treatment chamber pressure below the external atmospheric pressure before introducing the noble gas.
